(19) United States
(12) Patent Application Publication
(10) Pub. No.: US 2006/0052796 A1
(43) Pub. Date: Mar. 9, 2006

(54) Combined epithelial delaminator and inserter

(76) Inventors: Edward Perez, Redwood City, CA (US); Jay S. Daulton, Gilroy, CA (US); Paul K. Hsei, Huntington Beach, CA (US)

Correspondence Address: MORRISON & FOERSTER LLP, 755 PAGE MILL RD, PALO ALTO, CA, 94304-1018, US

(21) Appl. No.: 10/937,135

(22) Filed: Sep. 8, 2004

(51) Int. Cl. A61F 9/00

(52) U.S. Cl. 606/107

(57) ABSTRACT

An ocular device applicator that is a combined delaminating and inserting device configured to partially delaminate the corneal epithelium and to place a corrective ocular device onto the delaminated cornea under the partially delaminated epithelium. The ocular device applicator leaves at least a portion of the delaminated corneal epithelium attached to the cornea. The ocular device applicator comprises an edge and an ocular device holder. In some versions an ocular device may be secured in and/or released from the device by force applied in the holder, or by a releasable adhesive. The ocular device applicator may be oscillated to facilitate delamination of the corneal epithelium and placement of an ocular device.

### FIELD

The described devices and methods are useful in the field of ophthalmology. Described herein are applicators and methods of using applicators for introducing an ocular device beneath a corneal epithelium. The described devices and methods for using them involve separating or lifting corneal epithelium from the eye in a substantially continuous layer to form a flap or pocket. In particular, the devices and methods generally utilize a combined epithelial delaminator and ocular device inserter. The combined delaminator and inserter is configured to separate the epithelium from the cornea, e.g., between the epithelium and the corneal stroma (Bowman's membrane) in the region of the lamina lucida, and also to introduce an ocular device on the eye without the need for an additional inserter or an additional insertion step. The devices and methods described herein may be used as part of an ocular therapy including ocular corrective surgery and laser eye corrective surgery.

### BACKGROUND

Refractive surgery refers to a set of surgical procedures that change the native optical or focusing power of the eye. The result of these procedures often alleviates the need for glasses or contact lenses that an individual might otherwise be dependent on for clear sight. The majority of the focusing power in the human eye is dictated by the curvature of the air-liquid interface, where there is the greatest change in the index of refraction. This curved interface is the outer surface of the cornea. The refractive power of this interface accounts for approximately 70% of the total magnification of the eye. Light rays making up seen images pass through the cornea, the anterior chamber, the crystalline lens, and the vitreous humor before being focused on the retina to form an image. It is the magnifying power of this curved, air-corneal interface that provided the field of refractive surgery with the opportunity to surgically correct visual deficiencies.

Early refractive surgical procedures corrected nearsightedness by flattening the curvature of the cornea. The first largely successful procedure was called radial keratotomy (RK). RK was widely used during the 1970's and early 1980's where radially oriented incisions were made in the periphery of the cornea. These incisions reformed the peripheral cornea by causing it to bow outwards, consequently flattening the central optical zone of the cornea. This was fairly easy and thus, popular, but it rarely did more than lessen one's dependency on glasses or contract lenses.

A largely flawed and failed procedure called epikeratophakia was developed in the era of RK. It is now essentially an academic anomaly. Epikeratophakia provided a new curvature to the outer curvature of the cornea by grafting onto the cornea a thin layer of preserved corneal tissue. The processed corneal tissue is freeze-dried and during the process of freeze drying, the cornea is also ground to a specific curvature. The resulting lens was placed into the eye surgically. An annular 360° incision was placed into the cornea after completely removing the epithelium from where the epikeratophakic lens would sit. The perimeter of this lens would be inserted into the annular incision and held in place by a running suture. There were several problems with epikeratophakia: 1) the lenses remained cloudy until host stromal fibroblasts colonized the lens, which colonization possibly could take several months; 2) until migrating epithelium could grow over the incision site onto the surface of the lens, the interrupted epithelium was a nidus for infection; and 3) epithelium healing onto the surgical site sometimes moved into the space between the lens and the host cornea. Currently, epikeratophakia is limited in its use. It is now used in pediatric aphakic patients who are unable to tolerate very steep contact lenses.

Around the mid 1990's procedures that sculpt the cornea with lasers were sufficiently successful that they began to replace radial keratotomy. The first generation of laser ablation of the cornea was called photorefractive keratectomy (PRK). In PRK, an ablative laser (e.g., an excimer laser) is focused on the cornea to sculpt a new curvature into the surface. In PRK, the epithelium is destroyed when achieving a new outer surface curve. Over the ensuing post-operative days, the epithelium has to grow or heal back into place. This epithelial healing phase was problematic for most patients since the epithelially denuded and ablated cornea was painful. It is also initially difficult to see following PRK, and this “recuperative time” can last from days to a week or more.

A subsequent variation of PRK corneal laser ablation, LASIK, has become very popular. The LASIK procedure, also known as laser in situ keratomileusis, is currently synonymous in the public mind with laser vision correction. In LASIK, an outer portion (or chord-like lens-shaped portion) of the cornea (80 to 150 microns thick) is surgically cut from the corneal surface. This is performed by a device called a microkeratome. The microkeratome cuts a circular flap from the surface of the cornea, leaving the flap comprising both epithelial and corneal tissue hinged at one edge. This flap is reflected back and an ablative (excimer) laser is used to remove or to reform a portion of the exposed surgical bed. The flap is laid back into place. When this flap is laid back into place, the cornea achieves a new curvature because the flap conforms to the laser-modified surface. In this procedure, epithelial cells are not removed or harmed. The epithelial cells have simply been incised at the edge of this flap. When the flap is placed back onto the corneal bed, the epithelium heals back at the incision site. There is essentially no recuperative time and the results are almost immediate. Because there is very little surgical time (15 minutes for each eye) and because there are lasting and very accurate results, LASIK is currently considered the premier manner of performing refractive surgery.

The newest technique being evaluated in high volume refractive surgical practices and in some academic centers is a procedure called Laser Assisted Subepithelial Keratomileusis (LASEK). In LASEK, a “flap” is made of only epithelium. This layer of epithelium is lifted off the cornea in a manner similar to LASIK but using an ethanolic wash. The ablative laser is focused just on the surface of the denuded cornea (in the same manner as was done with PRK). However, this epithelial flap is left intact, i.e., the epithelium physical structure is not destroyed although cellular viability is largely destroyed. It is simply rolled back into place after formation of the re-curved anterior portion of the cornea, resulting in much less recuperative time than with PRK. Current methods of LASEK are not as good as LASIK but the results are better than with PRK.

The corneal epithelium is a multilayered epithelial structure typically about 50 μm in thickness. It is non-cornified. The outer cells are living, although they are squamous in nature. The basal epithelial cells are cuboidal and sit on the stromal surface on a structure known as Bowman's membrane. The basal cell layer is typically about 1 mil thick (0.001″). The basal cells produce the same keratins that are produced in the integument, i.e., skin. The basal epithelial cells express keratins 5 and 14 and have the potential to differentiate into the squamous epithelial cells of the corneal epithelium that produce keratins 6 and 9. The corneal epithelium has a number of important properties: 1) it is clear; 2) it is impermeable; 3) it is a barrier to external agents; and 4) it is a highly innervated organ. Nerves from the cornea directly feed into the epithelium, and thus, defects of this organ produce pain.

Epithelial cells are attached side-to-side by transmembrane molecules called desmosomes. Another transmembrane protein, the hemidesmosome, connects to collagen type 7 and is present on the basolateral surface of basal epithelial cells. Hemidesmosomes anchor epithelium to the underlying collagenous portion of the stroma. The junction between the epithelium and corneal stroma is referred to as basement membrane zone (BMZ).

When LASEK is performed, a physical well is placed or formed on the epithelium and filled with a selection of 20 percent ethanol and balanced salt solution. Contact with the solution causes the epithelial cells to lose their adherence at the BMZ, most likely by destroying a portion of that cell population. The epithelium is then raised by pushing the epithelium in a manner similar to striping a wall of paint. The exposed collagenous portion of the corneal stroma is then ablated to reshape its surface. A weakened epithelium is then rolled back into place to serve as a bandage. However, this “bandage” fails to restore the epithelium to its original state, i.e., it does not preserve the integrity of the epithelium, thereby reducing its clarity, impermeability to water, and barrier function. Furthermore, the ability of the epithelium to adhere to the corneal stromal surface is impaired.

### REFERENCES

Kiistala, U. (1972). “Dermal-Epidermal Separation. II. External Factors in Suction Blister Formation with Special Reference to the Effect of Temperature,” Ann Clin Res 4(4):236-246.

Azar et al. (2001). “Laser Subepithelial Keratomileusis: Electron Microscopy and Visual Outcomes of Flap Photorefractive Keratectomy,” Curr Opin Ophthalmol 12(4):323-328.

Beerens et al. (1975). “Rapid Regeneration of the Dermal-Epidermal Junction After Partial Separation by Vacuum: An Electron Microscopic Study,” J Invest Dermatol 65(6):513-521.

Willsteed et al. (1991). “An Ultrastructural Comparison of Dermo-Epidermal Separation Techniques,” J Cutan Pathol 18(1):8-12.

Van der Leun et al. (1974). “Repair of Dermal-Epidermal Adherence: A Rapid Process Observed in Experiments on Blistering with Interrupted Suction,” J Invest Dermatol 63(5):397-401.

Katz S I. (1984). “The Epidermal Basement Membrane: Structure, Ontogeny and Role in Disease,” Ciba Found Symp 108:243-259.

Green et al. (1996). “Desmosomes and Hemidesmosomes: Structure and Function of Molecular Components,” FASEB J 10(8):871-881.

None of the cited references shows or suggests my invention as described herein.

### SUMMARY

The description includes ocular device applicators for introducing an ocular device beneath a corneal epithelium. The device applicators include a) an edge configured to mechanically separate a layer of the corneal epithelium from a cornea while maintaining the epithelial layer in at least partial attachment to the cornea and b) an ocular device holder configured to hold an ocular device. The ocular device holder often is also configured place the ocular device onto the cornea, beneath the separated layer of the corneal epithelium. The ocular device holder secures the ocular device in the applicator until the ocular device is placed on the cornea. The ocular device holder may be further configured to replace the epithelial layer over the implanted ocular device after the ocular device has been placed onto the cornea. In one version of the ocular deice applicator described herein, the ocular device holder comprises a recessed region into which all or a part of the ocular device can fit.

Examples of ocular devices that may be inserted using the devices and methods described herein include any biocompatible ocular device, such as lenses (e.g. contact lenses, implantable lenses, etc.), filters (polarizers, diffraction filters, etc), inserts, and the like. The ocular device may also be included as part of the applicator.

At least one edge of the applicator may be adapted to delaminate the epithelial layer from the cornea. In one version, this edge is substantially blunt. In one version, the edge is rounded. In one version, at least part of the delaminating edge is formed by at least part of an ocular device to be inserted. In one version, at least part of the edge is stainless steel. In one version, the edge of the applicator is substantially dull. In one version, the edge of the applicator is rounded. Thus, the applicator may delaminate the epithelial layer by a non-cutting means. In one version, the applicator is spatula shaped, so that the edge configured to delaminate the cornea is located at the end region of the spatula shape.

The applicator may be configured to create a loose epithelial flap, so that the portion of the corneal epithelial layer mechanically separated by the applicator remains attached to the cornea for 10% to 50% of the edge of the separated epithelial layer. The applicator may be configured to create an epithelial flap, so that the portion of the corneal epithelial layer mechanically separated by the applicator remains attached to the cornea for 50% to 75% of the edge of the separated epithelial layer. The applicator may be configured to create an epithelial pocket, so that the portion of the corneal epithelial layer mechanically separated by the applicator remains attached to the cornea for 50% to 95% of the edge of the separated epithelial layer.

In one version, the applicator is configured so that the edge oscillates. Oscillation may help in separating the epithelial layer. For example, the applicator may oscillate the edge side to side, backwards or forwards, in a circular (or partially circular) motion, or some combination thereof. Oscillation may be in the plane of the applicator edge, or out of the plane of the applicator edge.

In one version, a region of the applicator has a low-friction surface. In particular, the portion of the applicator which contacts any of the delaminated epithelial layer is a low-friction surface. A low-friction surface reduces the likelihood of damage (e.g. tearing) to the delaminated epithelium as the applicator is used. For example, the surface may be coated with a substance which reduces friction (such as a biocompatible lubricant, diamond coating, etc). Other low-friction surfaces include polished surfaces.

The ocular device is releasably held in the ocular device holder. In one version, the ocular device is releasably held in the device holder by a releasable adhesive, such as a water-soluble material (e.g. a biocompatible soluble polymer such as polyvinylalcohol). In one version, the ocular device holder of the applicator is configured to apply positive or negative force to an ocular device in the ocular device holder. For example, the ocular device holder may include a channel fluidly connected to the holder so that positive or negative force (such pressure from gas or liquid) can be applied through the channel to secure or release an ocular device from the ocular device holder of the applicator.

Also described herein are kits for inserting an ocular device beneath a corneal epithelium. The kits contain a combined delaminating and inserting device for delaminating the corneal epithelium having an edge configured to delaminate an epithelial layer of an eye while maintaining the epithelial layer in at least partial attachment, and a holder configured to hold an ocular device. The kit also contains an ocular device held within the holder.

Also described herein are methods for delaminating an epithelial layer from an eye using a combined delaminating and inserting device. The method includes: lifting from the anterior corneal surface of an eye, a substantially continuous epithelial layer using a combined delaminating and inserting device. The combined epithelial delaminating and inserting device (also referred to as an ocular device applicator) has an edge and a holder, wherein the holder is configured to hold an ocular device.

Also described herein are methods of inserting an ocular device beneath a corneal epithelium using an ocular device applicator. The method includes: lifting from the anterior corneal surface of an eye a substantially continuous epithelial layer by inserting below the epithelium an ocular device applicator and inserting onto the anterior corneal surface an ocular device from the holder of the ocular device applicator. The ocular device applicator has an edge and a holder configured to hold an ocular device.

### BRIEF DESCRIPTION OF THE DRAWINGS

FIG. 1A is a top view of an ocular device applicator useful in separating the corneal epithelium and inserting an ocular device between the cornea and the corneal epithelium.

FIG. 1B is a side view of the ocular device applicator from FIG. 1A.

FIG. 1C is a bottom view of the ocular device applicator from FIGS. 1A and 1B.

FIG. 2A is a top view of another ocular device applicator.

FIG. 2B is a bottom view of the ocular device applicator from FIG. 2A.

FIG. 2C is a side view of the FIG. 2A applicator showing a lens to be placed.

FIG. 3A is bottom view of another ocular device applicator.

FIG. 3B is a perspective section through line L-L′ of FIG. 3A.

FIG. 4A shows a representational view of the top of an applicator similar to the applicator of FIG. 3.

FIG. 4B shows a representational view of the bottom of an applicator similar to the applicator of FIG. 3.

FIG. 5 is a representational view of the bottom of an applicator similar to the applicator shown in FIG. 4.

FIG. 6A is a schematic view of the top of an applicator and an applicator mount.

FIG. 6B is a schematic view of the bottom of the applicator shown in FIG. 6A.

FIG. 7A shows a view of a hand-held ocular device applicator attached to a mount and mount holder.

FIG. 7B shows details of the ocular device applicator region of the device shown in FIG. 7A.

FIG. 8 shows a histological section through a retina delaminated with one of the ocular device applicators described herein.

### DETAILED DESCRIPTION

The ocular device applicators described herein are combined corneal epithelial delaminators and ocular device inserters (hereinafter called “applicators” or “ocular device applicators”).

A continuous layer of corneal epithelium may be separated from or lifted from the anterior surface of the eye by applying various mechanical forces to this anterior surface, or to the basal cell layer, or to the junction between the basal cell layer and the Bowman membrane (the “lamina lucida”). The term “continuous” as used herein means “uninterrupted”. More or less epithelium may be separated from the cornea. For example, the devices and methods disclosed herein may be used to create a loose flap of corneal epithelium, leaving less than 50% (preferably between 10% and 50%) of the edge of the delaminated epithelium attached to the cornea. Similarly, a flap of corneal epithelium may be made from the corneal epithelium, leaving between 50% and 75% of the edge of the delaminated epithelium attached to the cornea. A half flap, or tight pocket, of delaminated corneal epithelium may also be formed by leaving between 50% and 95% of the edge of the delaminated epithelium attached to the cornea.

The applicators described herein may also be used to insert an ocular device onto the region of the cornea that has been delaminated of epithelium from the corneal stroma. In particular, the applicators described herein allow an ocular device to be inserted onto the delaminated cornea, beneath the epithelium that was separated from the cornea. The separated epithelium can then be placed or situated atop the inserted ocular device.

The term “ocular device” is intended to include any implantable ocular device, preferably ocular devices intended to modify, improve or correct vision in a patient in need thereof One such suitable ocular lens device to be used with the present invention is described in Application No. PCT/US01/22633 which is herein incorporated by reference in its entirety. Examples of ocular devices include: lenses (such as contact lenses, implantable lenses, etc.), filters (e.g. diffraction gratings, polarizers, etc.), implants (e.g. implants to reshape the eye surface), and the like.

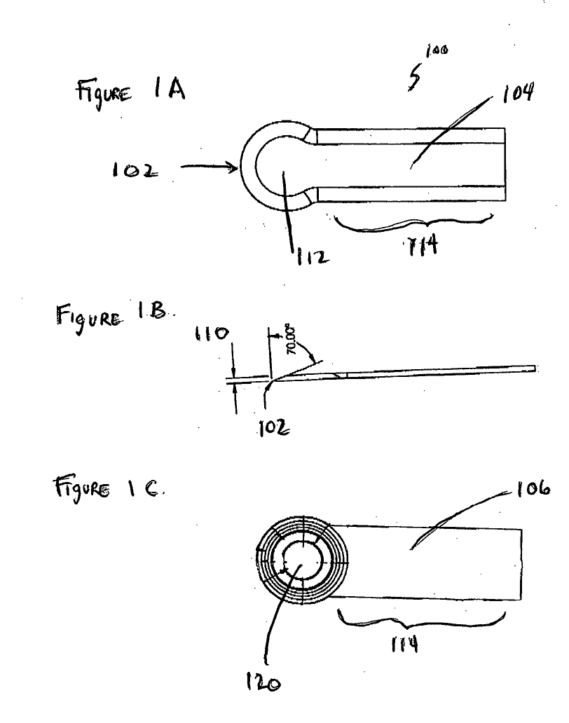

In a first variation of an applicator, the combined delaminator/inserter comprises a blunt tool 100 as is seen in FIG. 1. In general, these applicators have an elongated shape terminating in an edge region 102. Overall, this version of the applicator may be substantially flat, as shown in the profile of the applicator in FIG. 1B. This applicator includes a top 104, or upper, surface and a lower, or bottom, surface 106. The bottom surface 106 is shown in FIG. 1C. For the sake of convenience, the region of the applicator facing the delaminated corneal surface will be referred to as the bottom 106 surface of the applicator and the region of the applicator facing the delaminated corneal epithelium will be referred to as the top 104 surface. The applicator is shown as substantially flat, and having a uniform thickness 110 across its length, although other shapes (e.g. non-uniform thicknesses, “wedge” shapes, etc) are also intended to be encompassed by this description. FIG. 1C also illustrates an ocular device holder (or holding region), shown as a cavity 120 in the bottom surface into which at least a portion of an ocular device fits.

In operation, the applicator 100 may be attached to an applicator mount and/or a handle, so that it may be controlled by a user. The applicator mount may include or be connected to a driver motor in such a way that the edge or blunt tip region 102 moves it a repetitive, oscillatory motion that easily separates corneal epithelium from its underlying tissue without cutting that stromal tissue. In at least one variation of the device, the edge 102 moves in at least one of a side-to-side motion and an up-and-down motion. The edge may also be moved in a circular or semi-circular motion, for example, following a radius smaller than the diameter of the tip region.

The Applicator Edge Region

The edge 102 of the applicator is the region which mechanically interacts with the cornea to delaminate the epithelial region from the surface of the cornea. The edge region may therefore have any shape which facilitates this interaction. In cross-sectional profile, the edge region is shown as a wedge-shaped angle in FIG. 1B, but the edge 102 may be of other profiles, e.g., pointed, flattened or curved. The cross-sectional profile of the edge may be of any degree of bluntness, from the very blunt to very shap (approaching a knife edge). The cross-sectional profile of the edge may also vary over the length of the edge. Such choices are left to the designer at the time this teaching is taken and applied to the design of a tool for accomplishment of a specific task or procedure. For instance, the choice of a wide applicator 100 with a blunt tip will create a large epithelial pocket, for example, for installing a large contact lens in that pocket.

When the cross-sectional profile of the edge is generally wedge-shaped, the angle of the edge profile may also vary over a reasonable range. For example, in FIG. 1B, the edge is shown having an angle of 20° from the horizon (70° from the vertical). In one version, the edge profile ranges from 5° to greater than 45°. In one version, the angel is constant over the cross-sectional profile. In one version, the cross-sectional angle of the edge varies. For example, in one version, the portion of the edge closest to the bottom surface is be around 20°, while the angle decreases as the edge approaches the upper surface. In one version, the angle decreases so that there is no visible transition from the edge to the upper surface. In one version, the steepness of the edge profile increases as it approaches the upper surface. The transition between the edge region and the lower and upper surface may be blunt (e.g. smooth), or sharp (e.g. angular).

Functionally, the edge may be considered as having a bluntness appropriate to separate the epithelium from the cornea and produce a delaminated epithelial layer without corneal tissue attached. In one version, the delaminated epithelial layer has only an insignificant amount of corneal tissue attached. In one version, the delaminated epithelium has no corneal tissue attached.

The size and shape of the edge region (e.g. as shown in FIG. 1A, 102) may also vary. For example, the size and shape of the edge may be chosen based on the intended use of the applicator and the size of the flap or pocket desired. In FIG. 1, the edge region is shown as a semi-circle. Virtually any shape may be used that is capable of achieving the purposes described herein. Fore example, the edge region may be shovel-shaped, heart-shaped, rectangular, etc, or may simply be flat. The size of edge region (e.g. width) may also vary. Finally, the edge regions in FIGS. 1-7 are shown as substantially flat, however the edge region may be shaped, for example, to better conform to the slight curvature of the cornea. Other examples of edge regions that can be used for the inserter described herein are included in U.S. patent application Ser. No. 10/346,664 (filed Jan. 17, 2003) and U.S. provisional application 60/505,219 (filed Sep. 22, 2003), which are hereby incorporated by reference in their entirety.

Although the inserted may be described as flat or planar, these terms should be understood to specifically include shapes having a curvature in one axis (e.g. side to side) and in another axis (e.g. front to back) as appropriate to ease the mechanical separation of the epithelium from the corneal surface.

The edge of the inserter may penetrate the epithelium with and/or without additional help or manipulation of the surface of the epithelium. For example, the epithelium may be scored or otherwise disrupted (e.g. punctured, torn, etc.) before the applicator is used. In one version, the applicator may be used on an initially intact epithelium.

The edge of the inserter may be made of any material sufficient to withstand the force applied by the edge as it delaminates the epithelial layer from the cornea. Specifically, the edge region may be made of a metal, ceramic or polymer, and may also be coated with another similar or different material. The materials and/or coatings may enhance the ability of the edge to delaminate the epithelium from the cornea without damaging either the cornea or the epithelial cells. For example, the edge may be made of stainless steel which can be polished (e.g. electropolished) or coated. The edge material may also be made of the same material as the shaft region of the applicator, or it may be made from a different material. Applicators intended for use with living tissue are preferably made of a material which can be sterilized. The edge may also include a material which incorporates therapeutic properties (e.g. medicaments, growth factors, etc.) to assist the healing process, reduce pain, or to help the cornea in accepting the optical implant. For example, the edge region (or any region of the applicator) could release a medicament from a polymeric matrix while in contact with the eye.

In one version of the applicator, the edge is at least a region of the ocular device to be implanted. For example, the edge may be part of a lens made from a relatively stiff material, or a lens which is not yet fully hydrated. The lens is held by the ocular device holder and at least of region of the lens projects from the applicator and is used to delaminate the epithelial layer from the corneal surface. The lens is released from the applicator and secured into place after delaminating and positioning the lens above the corneal stroma. The applicator is then removed, leaving the lens in place (and re-hydrating the lens, if necessary).

The top surface 104 and the bottom surface 106 (including the shaft region 114) of the applicator may also affect applicator performance. In some versions of the applicator, the top 104 of the applicator contacts the newly delaminated corneal epithelium as the applicator is used. Thus, the surface properties of the top of the applicator can be adapted to enhance the function of the applicator. In particular, the top 104 of the applicator is adapted to reduce friction between the applicator and the delaminated corneal epithelial layer. For example, the top of the applicator may be made smooth by polishing, or by coating it with a material that reduces friction (e.g. a biocompatible lubricant). The top region may also comprise a material having a low coefficient of friction for the epithelial layer. In one version of the applicator, at least the top region of the applicator includes a diamond coating.

In general, the applicator may incorporate therapeutic materials (such as medicaments, etc), for example, to be released during use. All of the applicator, or portions of the applicator may be made of a material having therapeutic properties, or may be coated with (or infused with) a material having therapeutic properties.

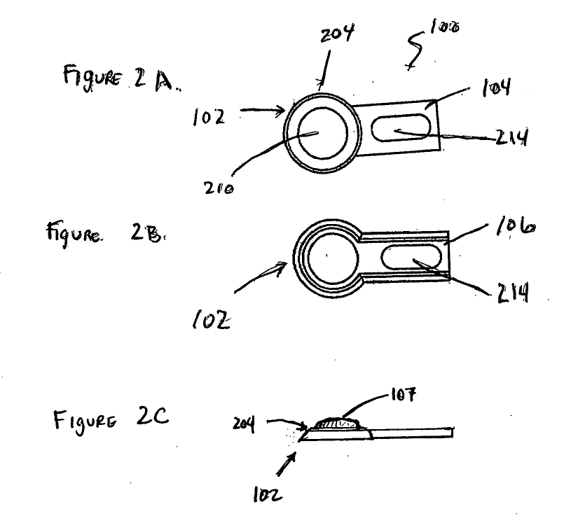

Friction between the top of the applicator and the delaminated epithelium may also be reduced by decreasing the overall bulk, volume, or size of the surface area which contacts the delaminated epithelium. FIGS. 2A and 2B show an applicator in which the edge is a ring 204. In this version, friction is reduced because the surface are of the top of the applicator has been reduced 210.

FIGS. 2A and 2B also show an attachment site 214 by which the applicator may be connected to an applicator mount. In one version the applicator is attached to an applicator mount at the end of the applicator furthest from the delaminating edge of the applicator. In this version, a driver is connected to the attachment site 214 on the applicator. The driver is configured to oscillate either the entire applicator or predominantly the edge region of the applicator.

Ocular Device Holder

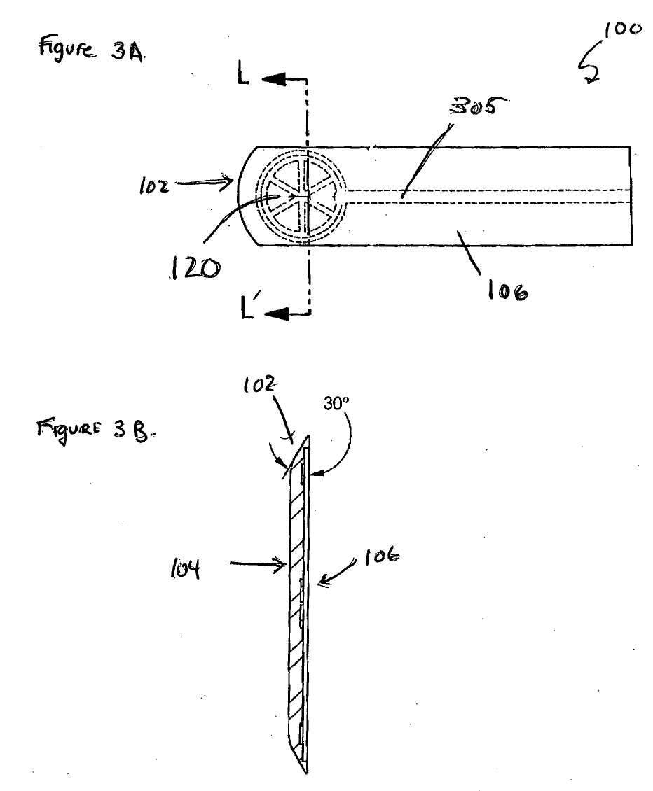

The ocular device applicator also includes an ocular device holder (“the holder”) to hold an implantable ocular device. FIG. 3A shows one version of an applicator holder, in which the holder is a recess in the bottom surface of the applicator 302. In general, the holder releasably secures an ocular device on (or in) the applicator. The holder releases a secured ocular device once the applicator has sufficiently separated the epithelium from the cornea so that the ocular device fits into the delaminated region.

The ocular device holder conforms to at least a portion of a region of the ocular device. In one version, the ocular device holder conforms to at least one outer surface of the ocular device. In one version, the ocular device is completely enclosed in the ocular device holder of the applicator. For example, in FIG. 3A, a lens would fit into the cavity formed by the holder 120 so that none of the lens projects beyond the plane of the bottom side 106. In FIGS. 1 and 3, the ocular device holder 120 is a cavity in the bottom of the applicator into which the ocular device fits. The holder is shown near the delaminating edge of the applicator 102, and the holder is surrounded by this edge on at least three sides. The holder does not have to be a recess in the bottom of the applicator. In one version, the holder projects from the bottom surface of the applicator. Placing the holder on or near the bottom of the applicator allows the applicator to easily deposit the ocular device after delaminating an appropriately-sized region of the cornea. In one version, the holder is a cavity in both the top and bottom of the applicator (for example, FIGS. 2A, 2B, and 2C). In particular, FIG. 2C shows a lens 107 situated within the holing region but extending from the top of the ring 204.

The ocular device holder holds the ocular device before and during delamination, and releases the ocular device after delamination is substantially complete. The ocular device may be held and/or released from the holder and by applying force to ocular device, or by using a releasable adhesive, or by a combination of both.

In one version, the ocular device is held in the ocular device holder by applying a vacuum. One or more channels 305 connect to the holder as shown in FIG. 3A. Force is applied to an ocular device in the holder through this channel. Negative force is applied to secure the ocular device in the holder (e.g. by drawing a vacuum) and positive force is applied to release the ocular device from the holder. For example, air pressure (or any other gas) may be applied through the channel to release the ocular device. In another example, fluid pressure (e.g. water or saline pushed through the channel) is applied to release the ocular device. Any fluid could be used to controllably hold and release the ocular lens in the applicator. Further, the channel may be used to apply useful substances (e.g. liquids such as saline, medicaments, etc.).

In one version the ocular device is held in the holder by a releasable adhesive. In particular, a dissolvable adhesive may be used. For example, in one version a water-soluble material secures the ocular device in the holder until it is ready to be released after insertion. Examples of water-soluble materials include, but are not limited to: polymers such as polyvinylalcohol, biopolymer such as hyaluronic acid (HA), and polysaccharides. Application of a fluid that releases the adhesive (e.g., saline or other beneficial fluid) causes the adhesive to dissolve or otherwise release, allowing implantation of the ocular device. Such a solution may be applied locally (e.g. through a channel 305) or over a larger area of the cornea.

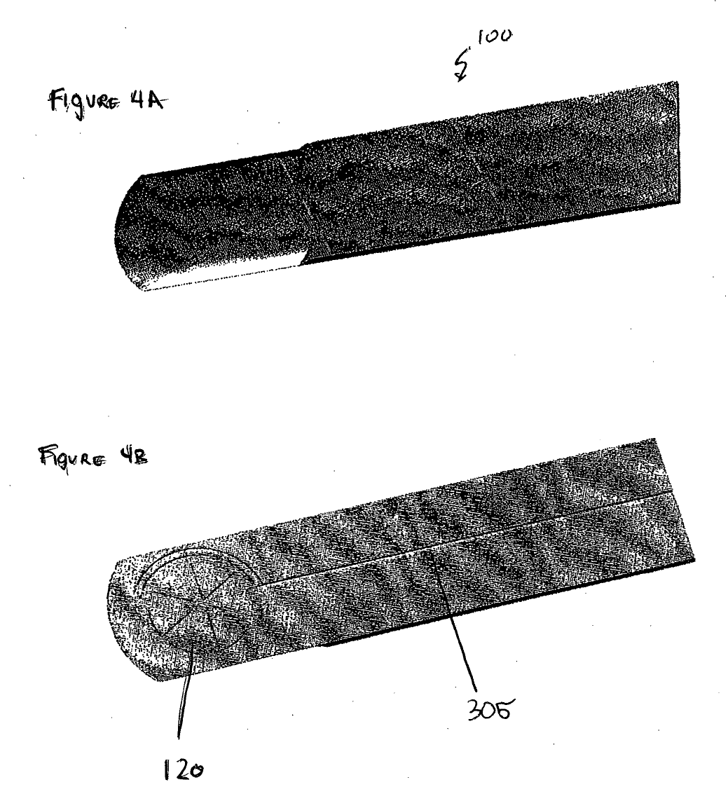

FIGS. 4A and 4B show a spatula-like applicator similar to the one shown in FIG. 3. In FIG. 4B, a channel 305 connects to the ocular device holder 120 at one end and also connects to a vacuum source and/or to a source of air or liquid (not shown) that are regulated to apply force to hold and/or to release an ocular device in the holder. The inner portion of the holder 120 includes additional channels that uniformly distribute the force over the ocular device held in the ocular device holder. The channel shown in FIG. 4B 305 is shown as open, but it is typically covered to allow pressure to be transferred to the holder.

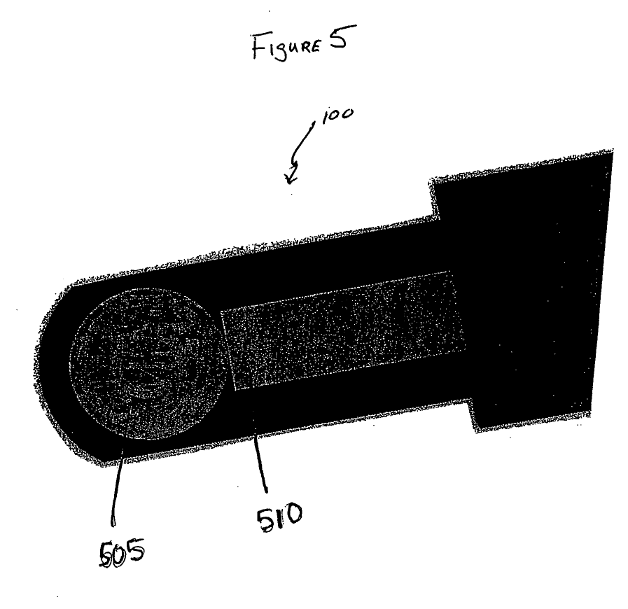

FIG. 5 shows the holder of FIG. 4 holding an ocular device 505 (a lens). The channel 305 connected to the holder has been sealed. In this example, the channel is sealed with an external covering (e.g. tape) 510. The channel may be incorporated within the inside of the applicator.

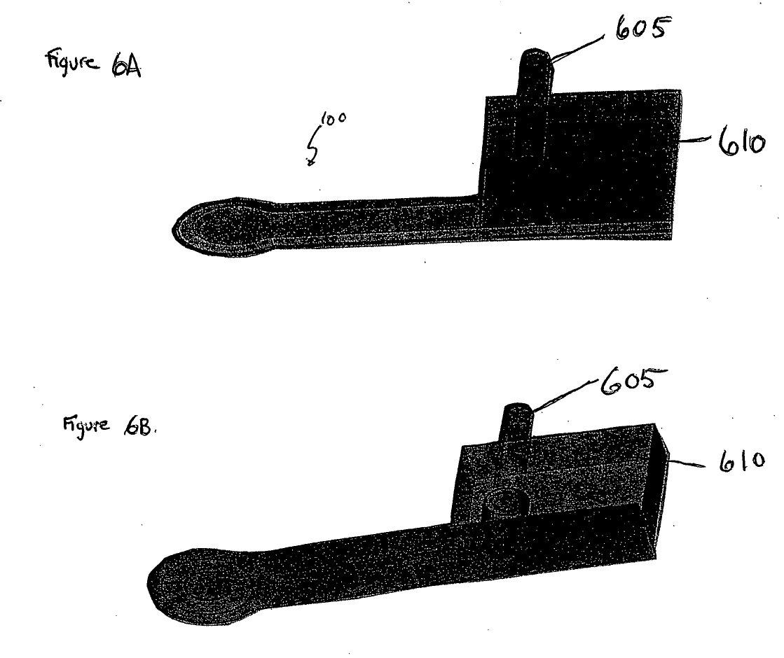

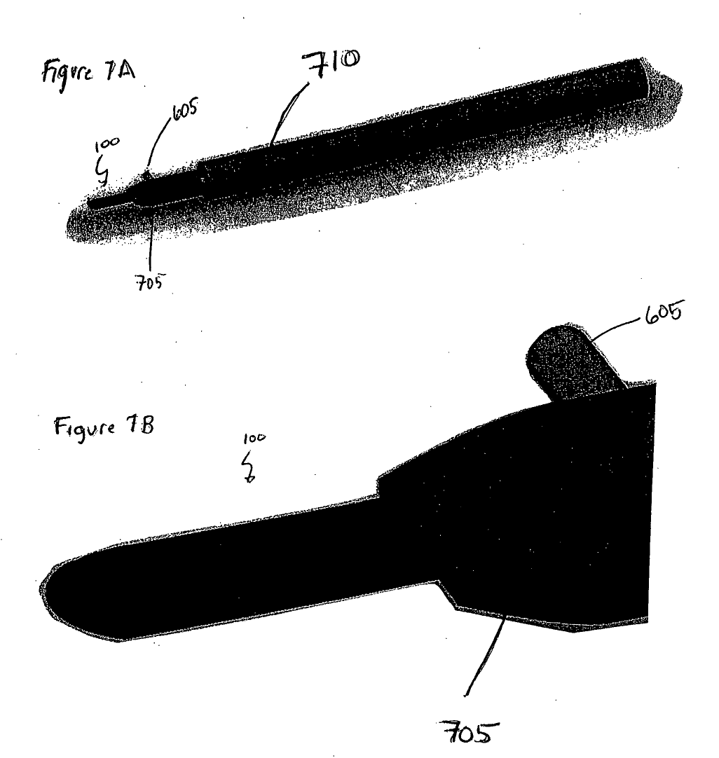

In operation, the applicator attaches to an applicator mount 610. The applicator mount may include additional components, such as a driver (e.g. to oscillate the edge of the applicator) or a port configured to connect to the channel to apply force (e.g. drawing a vacuum) to the holder. FIG. 6A shows a top view of an applicator connected to an applicator mount, and FIG. 6B shows a bottom view of the same applicator. The port 605 projects up out of the plane of the applicator for connection to a pressure source (e.g. via a tubing member). An alternative applicator mount is shown in FIG. 7B, in which the applicator is held by an applicator mount 705 also having a port, and further connecting to a handle 710 as shown in FIG. 7A.

The applicator may be fabricated either in separate parts (e.g. the edge, the holder, etc.) and assembled, or it may be fabricated as a single piece. For example, the applicator may be injection molded and/or micro-stamped into shape. The size of the applicator is chosen by the designer and depends in large part upon the intended purpose of the applicator, e.g. upon the device to be implanted and therefore should not be limiting. However, the applicator may have an overall thickness similar to the thickness of the basal cell layer, e.g., about ½ mil to 3.5 mils. (0.0005 to 0.0035″), but often about 1.0 mil to 3.0 mils (0.001 to 0.003″). For example, the edge of the applicator may have a thickness around 2.0 mils.

Although the procedure here is normally used to insert an ocular device beneath a substantially intact sheet of the epithelium, i.e., the portion of the epithelium that passes to the anterior side of the dissector is continuous, the device may be used in less elegant ways. For instance, the applicator may be used to remove selected portions of that membrane. Indeed, when this device is used in conjunction with a LASEK procedure, the epithelium may be removed in the form of a soft flap allowing for ease of replacement or re-positioning once any corneal laser remodeling is completed. In some instances it may be desirable to also apply heat to the anterior surface of the eye to enhance the mechanical epithelial delamination.

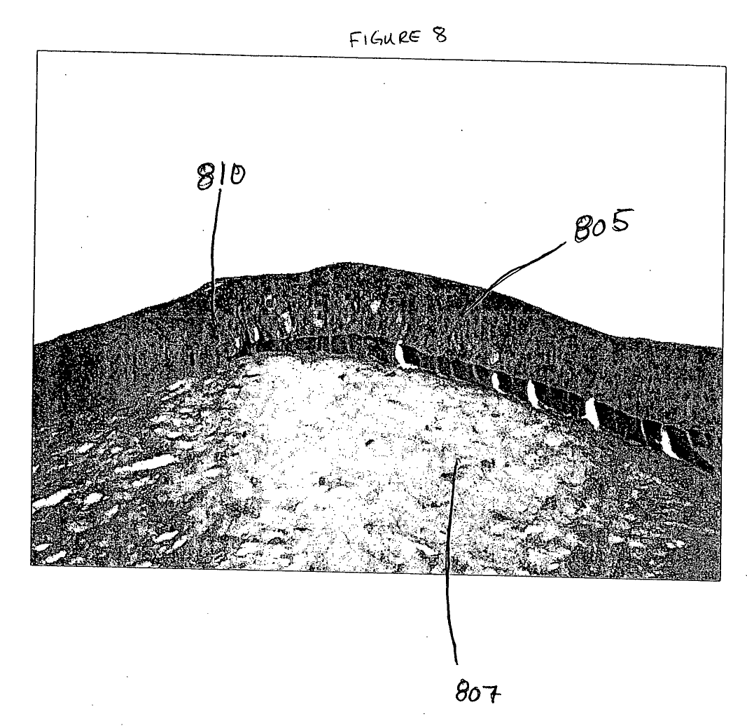

FIG. 8 shows an example of a cornea that has been delaminated using one of the inserters described in the present invention. The epithelial layer 805 has been cleanly separated from the cornea 807, leaving a portion of the separated layer attached the eye 810.

In one version, the applicator is used to delaminate the epithelium of an eye and to insert an ocular device onto the cornea between the corneal surface and the delaminated epithelium, which is placed on top of the ocular device. In one version of this method, an inserter as described herein is first placed near a portion of the epithelium on the outside of an eye. In one version the inserter is used in combination with a device (or devices) configured to position the inserter relative to the surface of the eye. In one version, the inserter is integral to such a device. Force may be applied to the inserter, which penetrates the epithelial layer (usually only once, though multiple cuts are also consistent with the methods and devices herein), and then moves across the eye beneath a portion of the epithelium but above the corneal surface (e.g. in the region of the lamina lucida). Thus, a portion of the epithelium is separated from the corneal stroma by the motion of the inserter, while maintaining the epithelial layer in at least partial attachment to the cornea. In one version the inserter (or a region of the inserter) is also oscillated to facilitate delamination of the epithelium as described herein. In one version, the ocular device is released from the inserter once a region of the cornea large enough to support the ocular device has been delaminated, and the inserter may be withdrawn, leaving the ocular device on the surface of the cornea. In one version the delaminated epithelium is a pocket which remains above at least a portion of the inserter as the ocular device is inserted. In another version the delaminated epithelium is at least partly folded away from the surface of the eye; after placement of the ocular device (or some other procedure, e.g. laser corrective surgery), the delaminated epithelium is replaced.

The epithelial delaminating methods herein described may also be used in conjunction with corneal reshaping procedures or procedures that do not involve placement of ocular lens devices on the surface of the eye. Specifically, the disclosed procedure may be used to prepare an epithelial pocket or a flap, often with an attached hinge. For example, a corneal reshaping procedure may be performed and the corneal flap replaced.

The structure and physiologic properties for my invention, as well as certain of the benefits particular to the specific variations of this epithelial delaminating device, have been described. This manner of describing the invention should not, however, be taken as limiting the scope of the invention in any way.

We claim:

1. An ocular device applicator for introducing an ocular device beneath a corneal epithelium comprising:
an edge configured to mechanically separate a layer of the corneal epithelium from a cornea while maintaining the epithelial layer in at least partial attachment to the cornea; and
an ocular device holder configured to place the ocular device beneath the layer of the corneal epithelium upon the cornea, and to hold the ocular device with the applicator until such placement.

2. The applicator of claim 1 wherein the applicator is configured to replace the epithelial layer onto the ocular device after placement of the ocular device onto the cornea.

3. The applicator of claim 1 wherein the ocular device holder further comprises a recessed region.

4. The applicator of claim 3 wherein the recessed region is configured to hold the entire ocular device.

5. The applicator of claim 1 wherein the applicator is spatula-like in shape.

6. The applicator of claim 1 wherein the applicator is further configured to produce an attached epithelial layer having an epithelial edge that is attached to the cornea for 10% to 50% of the length of the epithelial edge.

7. The applicator of claim 1 wherein the applicator is further configured to produce an attached epithelial layer having an epithelial edge that is attached to the cornea for 50% to 75% of the length of the epithelial edge.

8. The applicator of claim 1 wherein the applicator is further configured to produce an attached epithelial layer having an epithelial edge that is attached to the cornea for 50% to 95% of the length of the epithelial edge.

9. The applicator of claim 1 wherein the applicator is further configured to oscillate the edge.

10. The applicator of claim 9 wherein the applicator is configured to circularly oscillate the edge.

11. The applicator of claim 1 wherein the edge is rounded.

12. The applicator of claim 1 wherein the edge is substantially dull.

13. The applicator of claim 1 wherein at least a portion of the applicator comprises a low-friction surface.

14. The applicator of claim 13 wherein at least a portion of the applicator has a low-friction coating.

15. The applicator of claim 14 wherein the low-friction coating comprises a diamond coating.

16. The applicator of claim 1 wherein the ocular device holder is configured to allow positive and/or negative force to be applied to an ocular device held in the ocular device holder.

17. The applicator of claim 1 wherein the ocular device holder is fluidly connected to a channel configured to pass positive and/or negative force to the ocular device holder.

18. The applicator of claim 17 wherein the ocular device holder is further configured to release an ocular device by applying a positive force through the channel.

19. The applicator of claim 17 wherein the ocular device holder is further configured to secure an ocular device in the ocular device holder by applying a negative force though the channel.

20. The applicator of claim 17 wherein the channel allows pressure to be applied to an ocular device held in the ocular device holder.

21. The applicator of claim 20 wherein the pressure is air pressure.

22. The applicator of claim 1 wherein the edge has a rounded tip.

23. The applicator of claim 1 wherein the edge is substantially blunt.

24. The applicator of claim 1 wherein the edge is rounded.

25. The applicator of claim 1 wherein the edge is stainless steel.

26. The applicator of claim 1 further comprising an ocular device secured in the ocular device holder.

27. The applicator of claim 26 wherein the edge comprises at least a region of the ocular device

28. The applicator of claim 26 wherein the ocular device is secured in the ocular device holder by a releasable adhesive.

29. The applicator of claim 28 wherein the releasable adhesive is a water-soluble polymer.

30. The applicator of claim 29 wherein the releasable adhesive is polyvinylalcohol.

31. A combined delaminating and inserting device for delaminating at least a portion of the corneal epithelium from a cornea and placing an ocular device on the cornea comprising:
an edge configured to delaminate an epithelial layer of an eye;
an upper surface adjacent to the edge, wherein the upper surface comprises a low-friction surface; and,
an ocular device holder configured to hold an ocular device.

32. A combined delaminating and inserting device for delaminating at least a portion of the corneal epithelium and placing an ocular device on the delaminated cornea comprising:
an ocular device holder configured to releasably hold the ocular device;
an edge configured to mechanically separate a layer of the corneal epithelium from a cornea while maintaining the epithelial layer in at least partial attachment to the cornea; and,
an ocular device held within the holder.

33. The delaminating and inserting device of claim 32 wherein the edge is comprised of at least a region of the ocular device.

34. An ocular device applicator for introducing an ocular device beneath a corneal epithelium comprising:
an edge configured to mechanically separate a layer of the corneal epithelium from a cornea while maintaining the epithelial layer in at least partial attachment to the cornea;
an ocular device holder configured to place the ocular device beneath the layer of the corneal epithelium upon the cornea, and to hold the ocular device with the applicator until such placement; and
an ocular device held within the ocular device holder.

35. A kit for inserting an ocular device beneath a corneal epithelium comprising:
a combined delaminating and inserting device for delaminating the corneal epithelium and placing an ocular device on the delaminated cornea comprising an edge configured to delaminate an epithelial layer of an eye while maintaining the epithelial layer in at least partial attachment, and a holder configured to hold an ocular device; and
an ocular device held within the holder.

36. A method for delaminating an epithelial layer from an eye using a combined delaminating and inserting device comprising:
lifting from the anterior corneal surface of an eye a substantially continuous epithelial layer using a combined delaminating and inserting device comprising an edge and a holder, wherein the holder is configured to hold an ocular device.

37. A method of inserting an ocular device beneath a corneal epithelium using a combined delaminating and inserting device comprising:
lifting from the anterior corneal surface of an eye a substantially continuous epithelial layer by inserting below the epithelium a combined delaminating and inserting device comprising an edge and a holder configured to hold an ocular device; and
inserting onto the anterior corneal surface an ocular device from the holder of the combined delaminating and inserting device.

38. The method of claim 37 further comprising replacing the epithelial layer over the inserted ocular device.

39. The method of claim 37 further comprising removing the delaminating and inserting device.

40. The method of claim 37 wherein the step of lifting from the anterior corneal surface a substantially continuous epithelial layer further comprises maintaining at least a portion of the epithelial layer attached to the cornea.

41. The method of claim 37 wherein the step of lifting from the anterior corneal surface of a substantially continuous epithelial layer further comprises the step of inserting below the epithelium an oscillating combined delaminating and inserting device.
